(19) United States
(12) Patent Application Publication
(10) Pub. No.: US 2009/0215826 A1
(43) Pub. Date: Aug. 27, 2009

(54) SOLID FORMS OF A PYRROLIDINE-3,4-DICARBOXAMIDE DERIVATIVE

(76) Inventors: Jean-Michel Adam, Rosenau (FR); Andre Bubendorf, Uffheim (FR); Annette Deynet-Vucenovic, Loerrach (DE); Pascal Dott, Rixheim (FR); Alexander Glomme, Birsfelden (CH); Olaf Grassmann, Loerrach (DE); Wolfgang Haap, Loerrach (DE); Martin Kuentz, Muttenz (CH); Roland Meier, Ueken (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 12/388,536

(22) Filed: Feb. 19, 2009

Related U.S. Application Data
(63) Continuation of application No. 12/113,997, filed on May 2, 2008, now abandoned.

(30) Foreign Application Priority Data
May 10, 2007 (EP) 07107956.0

(51) Int. Cl. A61K 31/444; C07D 401/14

(52) U.S. Cl. 514/333; 546/256

(57) ABSTRACT

The invention is concerned with crystalline forms or amorphous forms of a pyrrolidine-3,4-dicarboxamide derivative, which is useful as an active ingredient of medicaments for the diseases which can be treated by the coagulation factor Xa inhibitors.

### PRIORITY TO RELATED APPLICATION(S)

This application is a continuation of U.S. application Ser. No. 12/113,997, filed May 2, 2008, which claims the benefit of European Patent Application No. 07107956.0, filed May 10, 2007. The entire contents of the above-identified applications are hereby incorporated by reference.

### BACKGROUND OF THE INVENTION

The present invention relates to new solid forms of a pyrrolidine-3,4-dicarboxamide derivative useful as an inhibitor of the coagulation factor Xa. (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide} of formula (I):

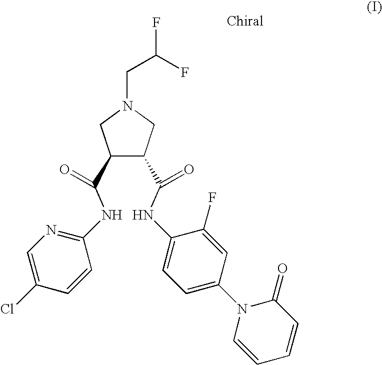

which is disclosed in WO2005/092881, is an inhibitor of the coagulation factor Xa. This compound consequently influences both platelet activation which is induced by this factor and plasmatic blood coagulation. Therefore, this compound inhibits the formation of thrombin and can be used for the treatment and/or prevention of thrombotic disorders, such as, among others, arterial and venous thrombosis, deep vein thrombosis, peripheral arterial occlusive disease (PAOD), unstable angina pectoris, myocardial infarction, coronary artery disease, pulmonary embolism, stroke (cerebral thrombosis) due to atrial fibrillation, inflammation and arteriosclerosis. Moreover, this compound can also be used in the treatment of acute vessel closure associated with thrombolytic therapy and restenosis, e.g. after transluminal coronary angioplasty (PTCA) or bypass grafting of the coronary or peripheral arteries and in the maintenance of vascular access patency in long term hemodialysis patients. In addition, this compound has an effect on tumor cells and prevent metastases. It can therefore also be used as antitumor agents.

The present invention is based on the discovery that certain new crystalline forms of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide} or amorphous forms thereof are suitable for preparing a pharmaceutical formulation.

### SUMMARY OF THE INVENTION

The present invention relates to new solid forms of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}, of formula (I):

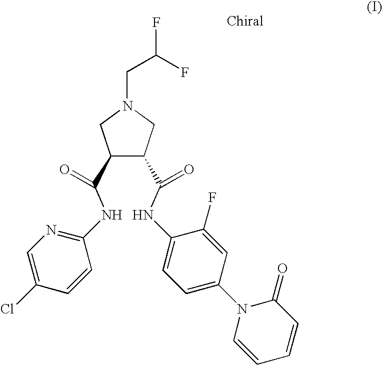

which is useful as an active ingredient in pharmaceutical compositions for diseases which can be treated by coagulation factor Xa inhibitors.

The specific crystalline forms of the present application are herein referred to as “crystalline form A” and “crystalline form B”.

Thus, the present invention relates to crystalline form A of the compound of formula (I), which is characterized by an X ray powder diffraction pattern comprising at least three, preferably five, more preferably seven of 2[theta] values selected from the group consisting of approximately 5.4, approximately 8.3, approximately 9.9, approximately 10.8, approximately 14.4, approximately 16.6, approximately 18.6, approximately 19.9, approximately 21.0, approximately 21.7, approximately 22.9 and approximately 26.0.

The present invention also relates to crystalline form B of the compound of formula (I), which is characterized by an X ray powder diffraction pattern comprising at least three, preferably five, more preferably seven of 2[theta] values selected from the group consisting of approximately 7.4, approximately 8.6, approximately 9.4, approximately 11.4, approximately 15.0, approximately 17.2, approximately 17.8, approximately 18.3, approximately 20.7 and approximately 27.8.

The present invention also relates to a crystalline form, consisting essentially of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide} and acetic acid.

The present invention also relates to amorphous forms of the compound of formula (I), which is characterized by an X ray powder diffraction pattern lacking a Bragg diffraction peak. These amorphous forms are also characterized by an X ray powder diffraction pattern comprising one or more amorphous halos.

The present invention also relates to pharmaceutical compositions comprising the crystalline form(s) mentioned above or the above mentioned amorphous compounds of formula (I) and a pharmaceutically acceptable excipient.

The present invention also relates to the crystalline form(s) mentioned above or the above mentioned amorphous compounds of formula (I) for use as a therapeutically active substance, especially as a therapeutically active substance for the treatment and/or prophylaxis of diseases which are associated with the coagulation factor Xa, particularly as therapeutically active substances for the treatment and/or prophylaxis of thrombotic disorders, arterial thrombosis, venous thrombosis, deep vein thrombosis, peripheral arterial occlusive disease, unstable angina pectoris, myocardial infarction, coronary artery disease, pulmonary embolism, stroke due to atrial fibrillation, inflammation, arteriosclerosis, acute vessel closure associated with thrombolytic therapy or restenosis, and/or tumor.

The present invention also relates to a use of the crystalline form(s) mentioned above or the above mentioned amorphous forms of the compound of formula (I) for the preparation of pharmaceutical compositions for the therapeutic and/or prophylactic treatment of diseases which are associated with coagulation factor Xa, particularly for the therapeutic and/or prophylactic treatment of thrombotic disorders, arterial thrombosis, venous thrombosis, deep vein thrombosis, peripheral arterial occlusive disease, unstable angina pectoris, myocardial infarction, coronary artery disease, pulmonary embolism, stroke due to atrial fibrillation, inflammation, arteriosclerosis, acute vessel closure associated with thrombolytic therapy or restenosis, and/or tumor.

### BRIEF DESCRIPTION OF THE DRAWINGS

FIG. 1 shows a XRPD (X-Ray Powder Diffraction) pattern of form A of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}.

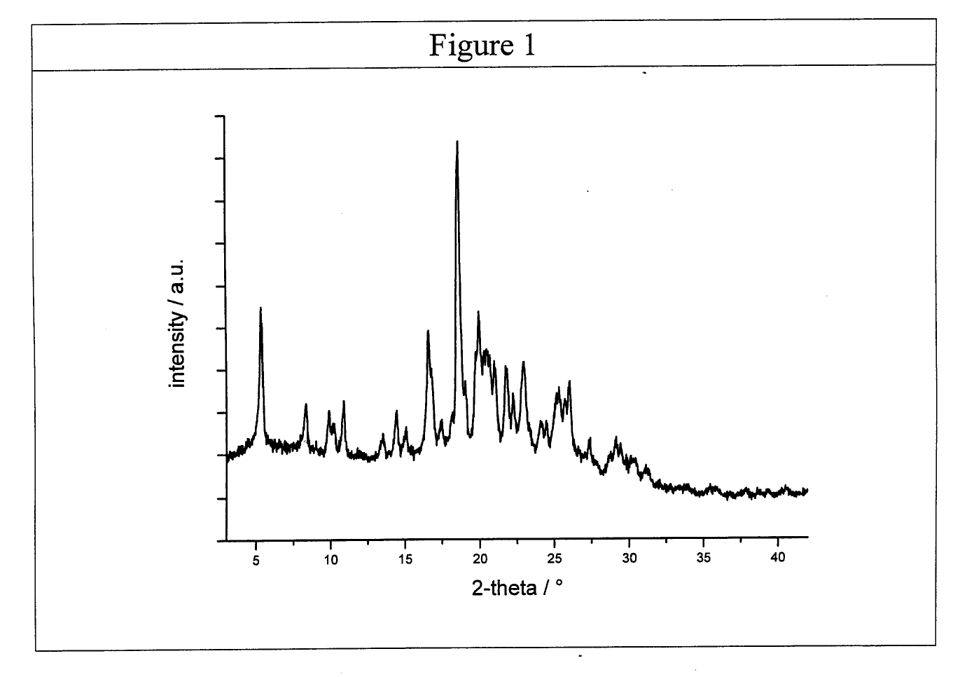

FIG. 2 shows an IR (InfraRed spectroscopy) spectrum of form A of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}.

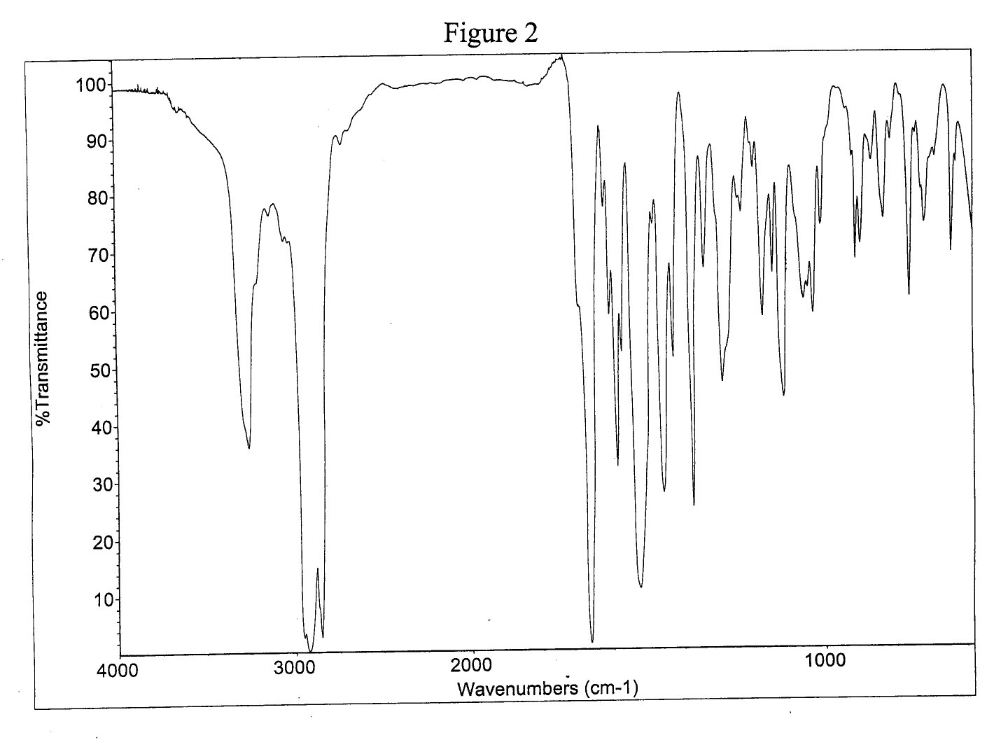

FIG. 3 shows an Raman (Raman spectroscopy) spectrum of form A of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}.

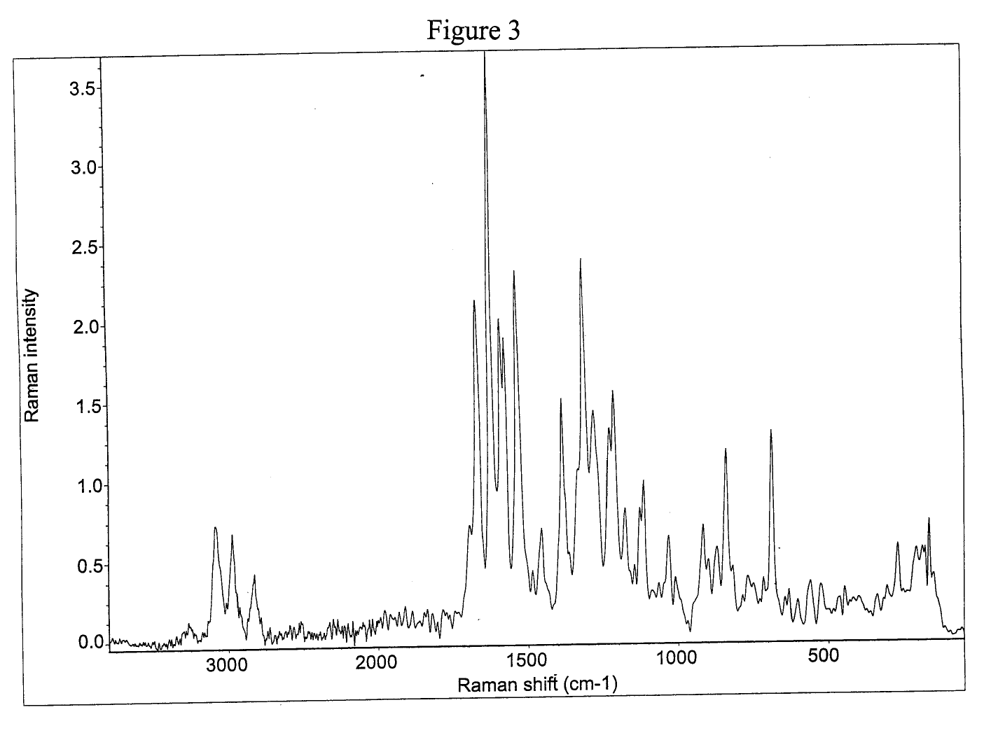

FIG. 4 shows a DSC (Differential Scanning Calorimetry) curve of form A of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}.

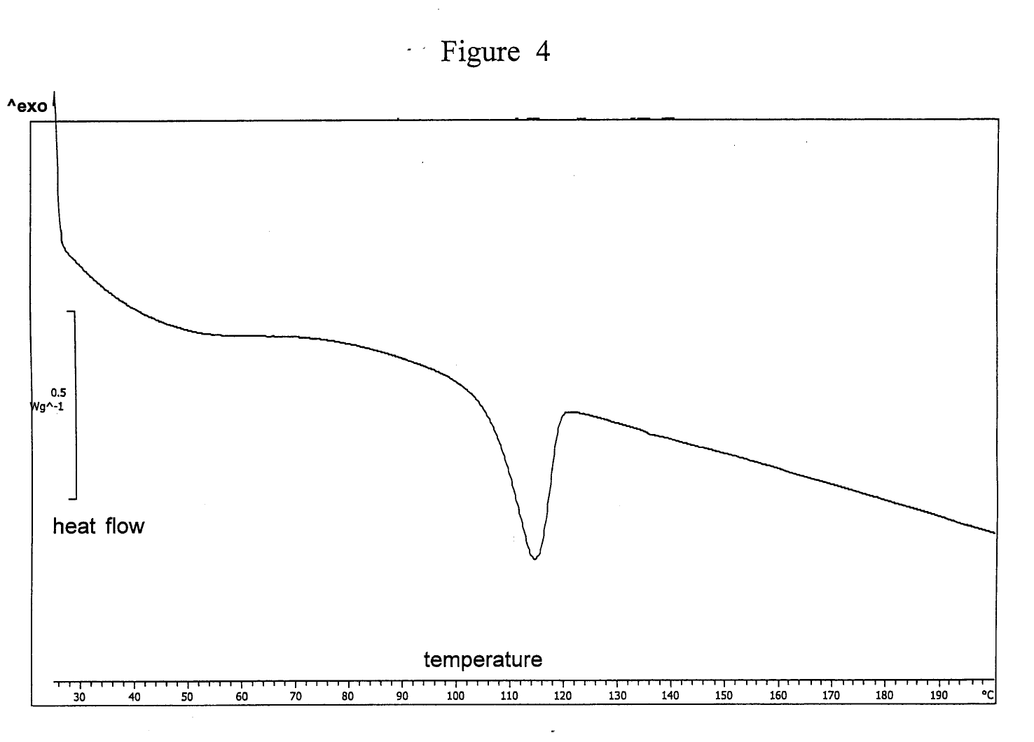

FIG. 5 shows a TGA (ThermoGravimetric Analysis) curve of form A of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}.

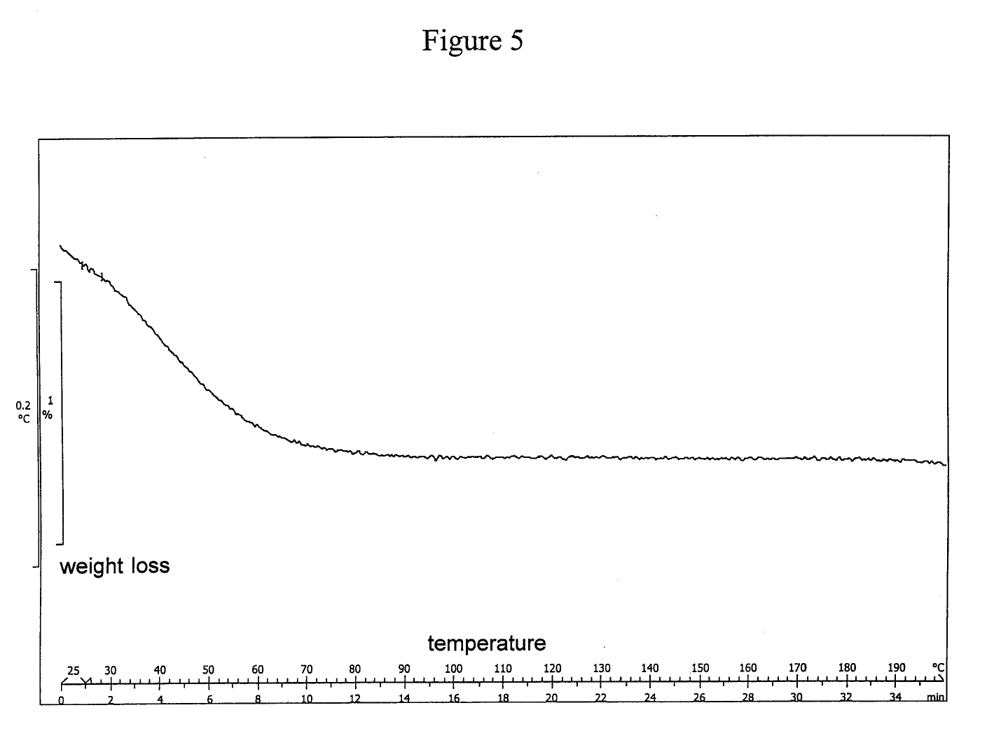

FIG. 6 shows a XRPD (X-Ray Powder Diffraction) pattern of form B of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}.

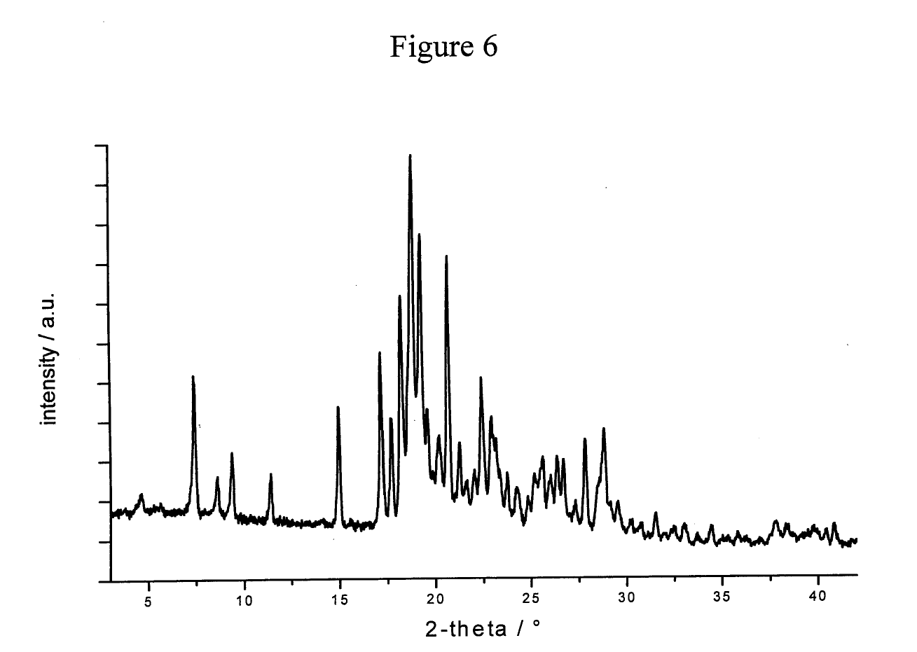

FIG. 7 shows an IR (InfraRed spectroscopy) spectrum of form B of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}.

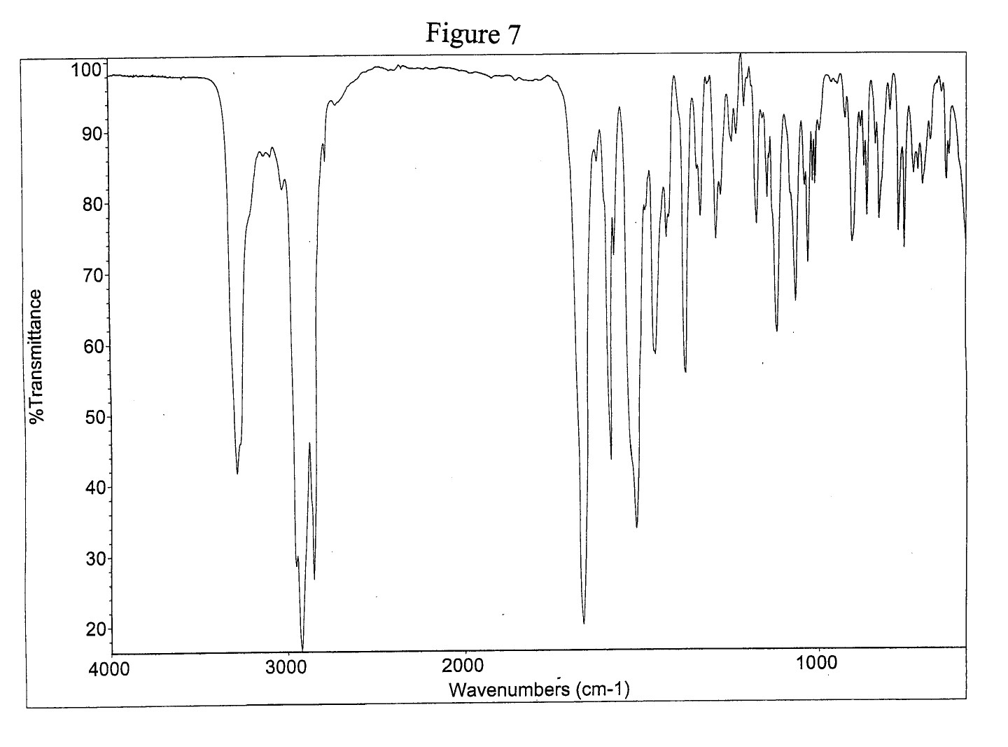

FIG. 8 shows an Raman (Raman spectroscopy) spectrum of form B of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}.

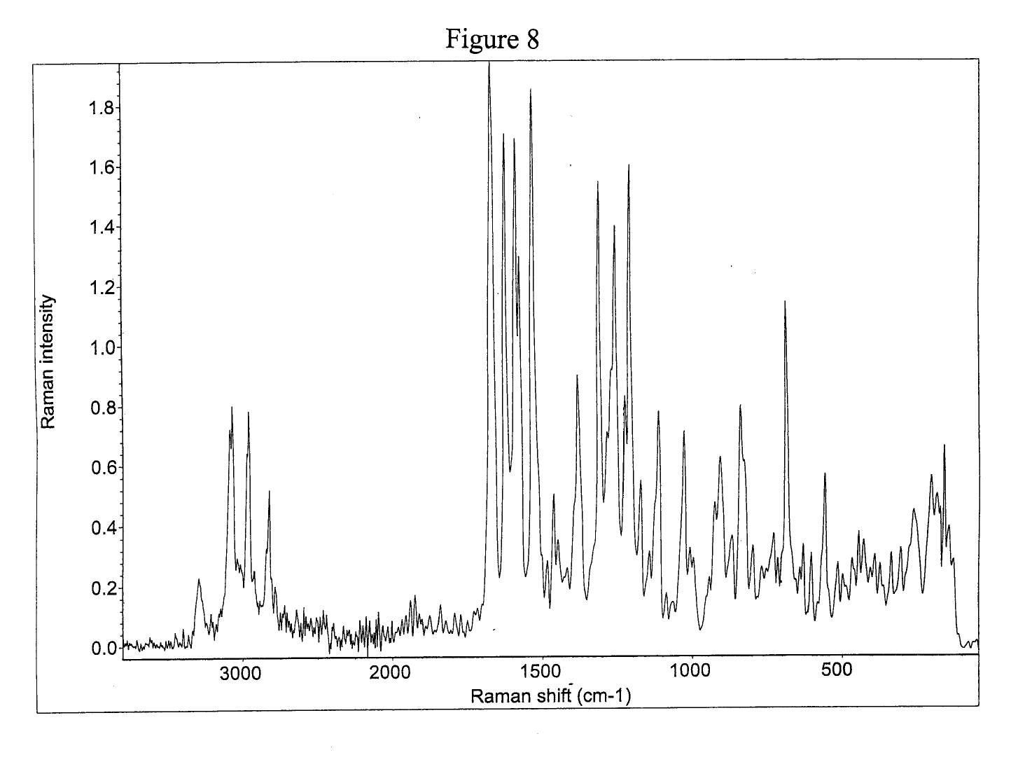

FIG. 9 shows a DSC (Differential Scanning Calorimetry) curve of form B of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}.

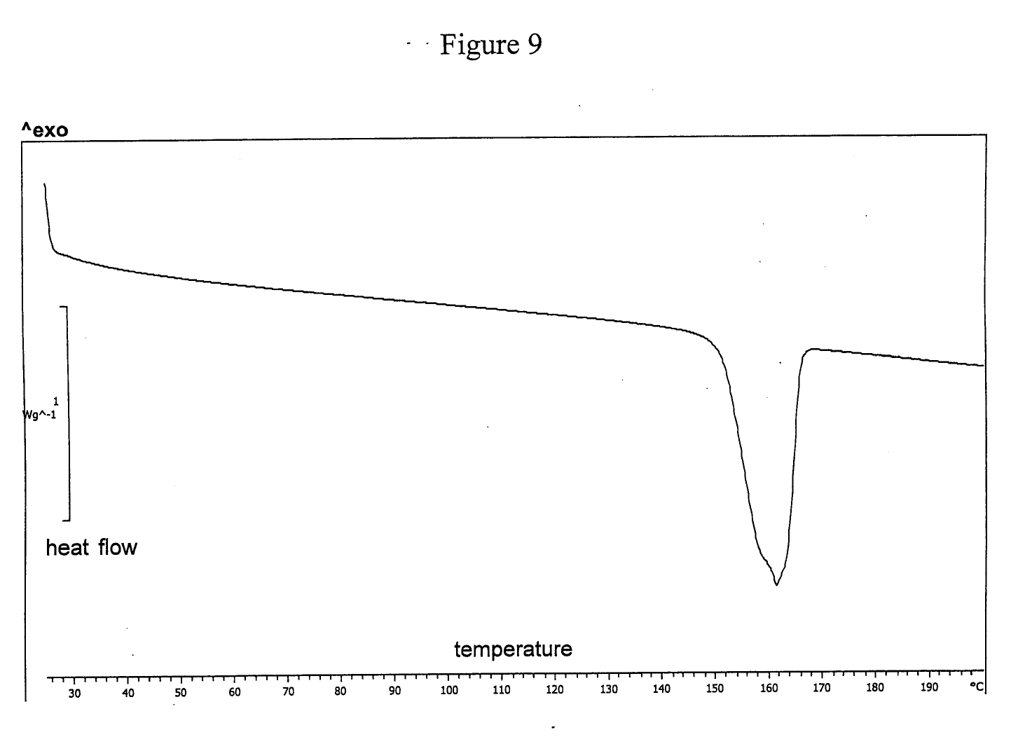

FIG. 10 shows a TGA (ThermoGravimetric Analysis) curve of form B of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}.

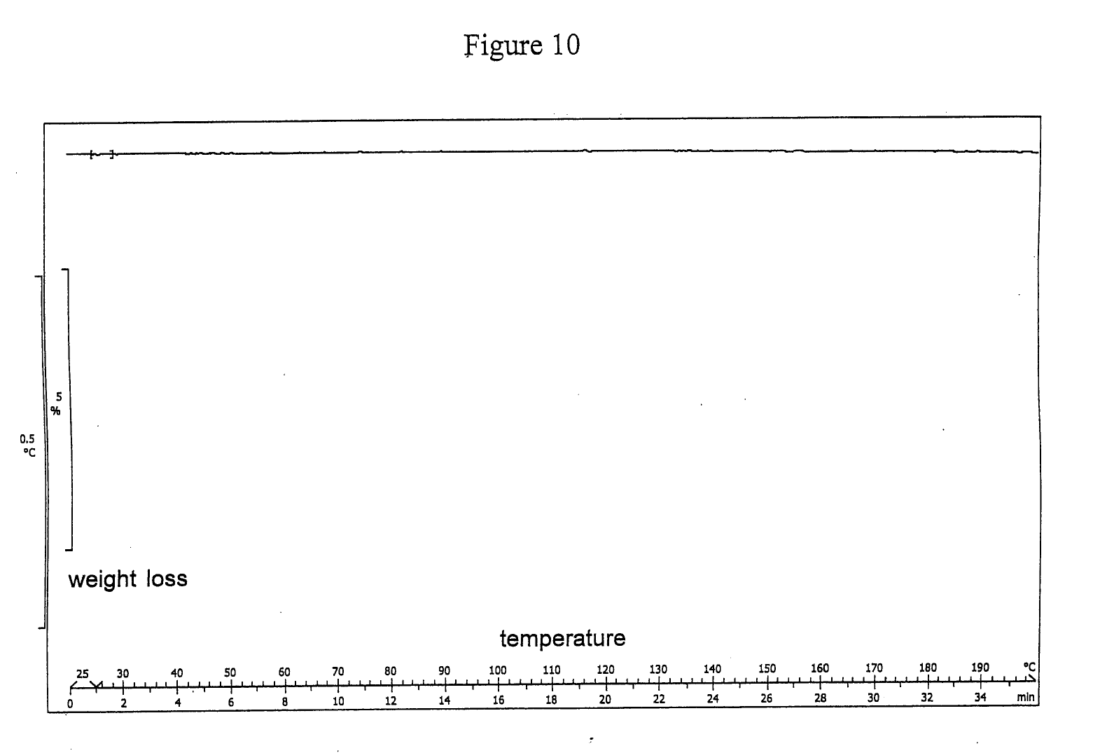

FIG. 11 shows a XRPD (X-Ray Powder Diffraction) pattern of the amorphous form of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}.

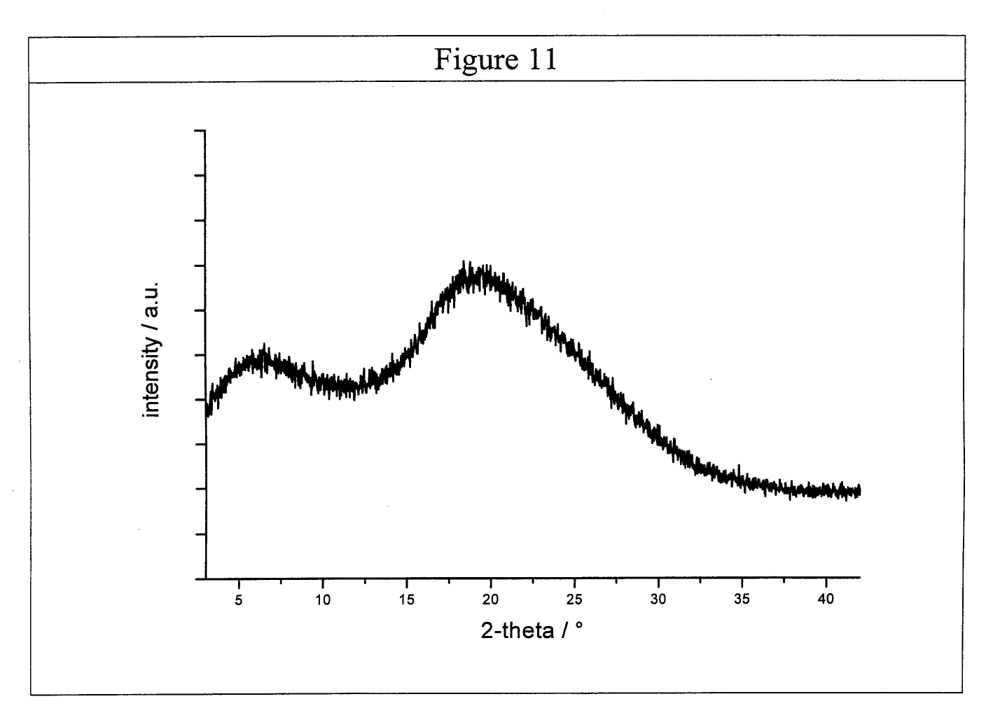

FIG. 12 shows an IR (InfraRed spectroscopy) spectrum of the amorphous form of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}.

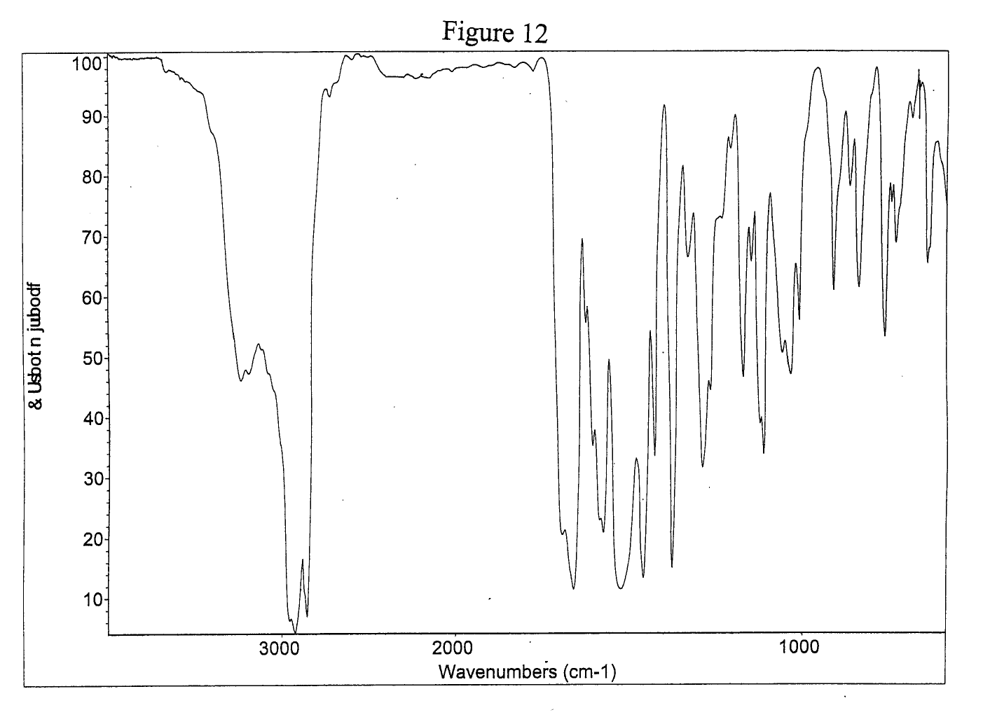

FIG. 13 shows an Raman (Raman spectroscopy) spectrum of the amorphous form of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}.

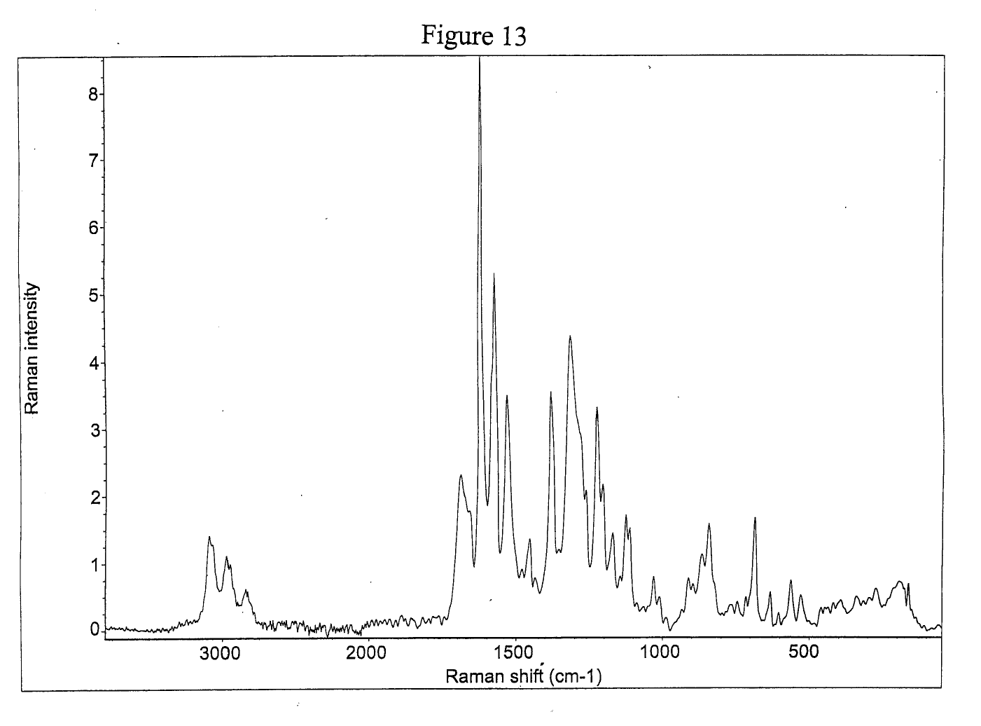

FIG. 14 shows a DSC (Differential Scanning Calorimetry) curve of the amorphous form of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}.

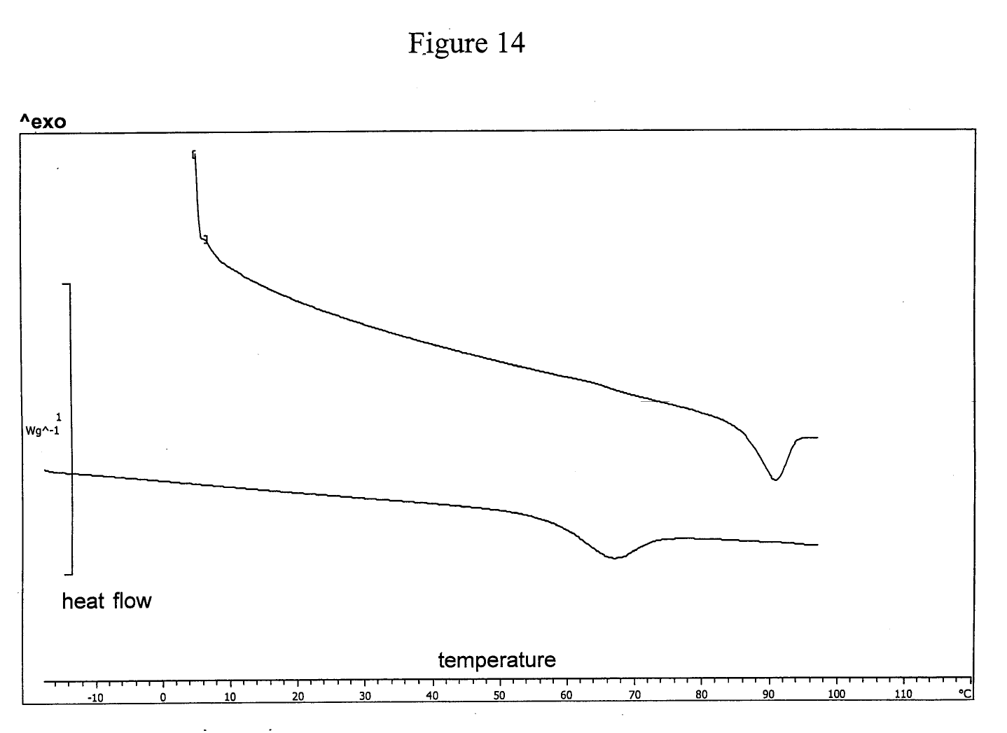

FIG. 15 shows a TGA (ThermoGravimetric Analysis) curve of the amorphous form of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}.

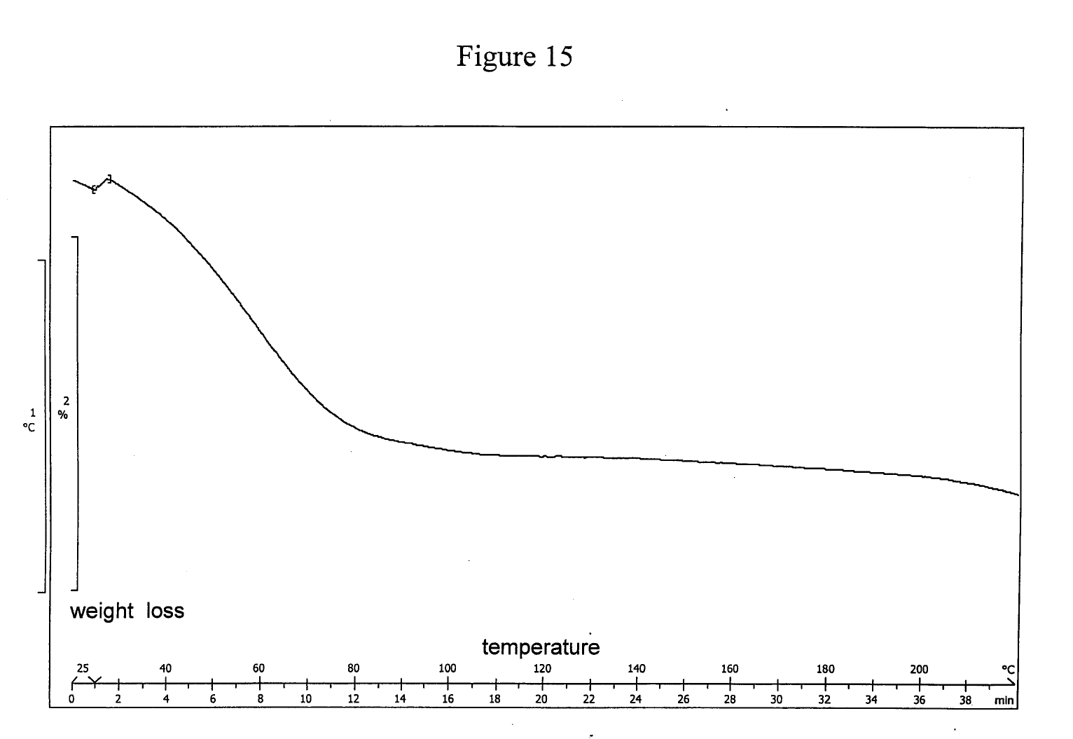

FIG. 16 shows a DVS (Dynamic Vapor Sorption) isotherm of the amorphous form of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}.

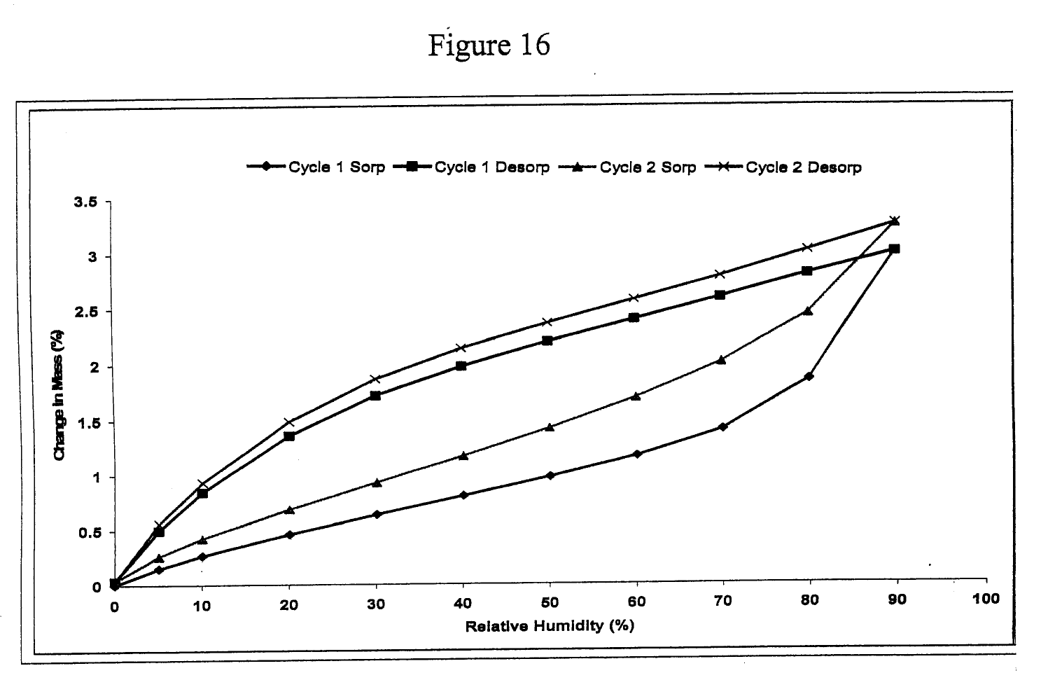

FIG. 17 shows a XRPD (X-Ray Powder Diffraction) pattern of a crystalline form, consisting of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide} and acetic acid.

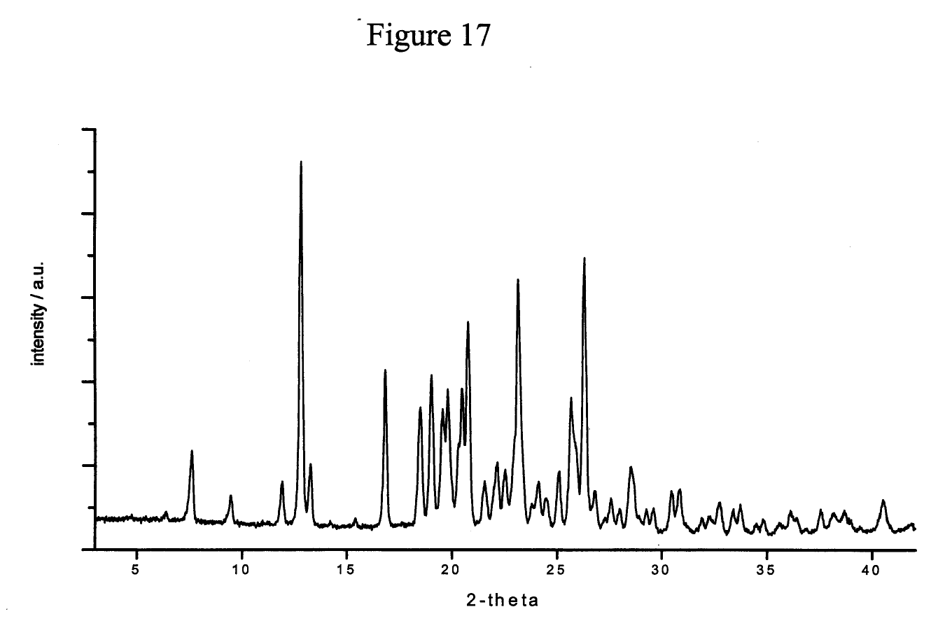

FIG. 18 shows an IR (InfraRed spectroscopy) spectrum of a crystalline form, consisting of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide} and acetic acid.

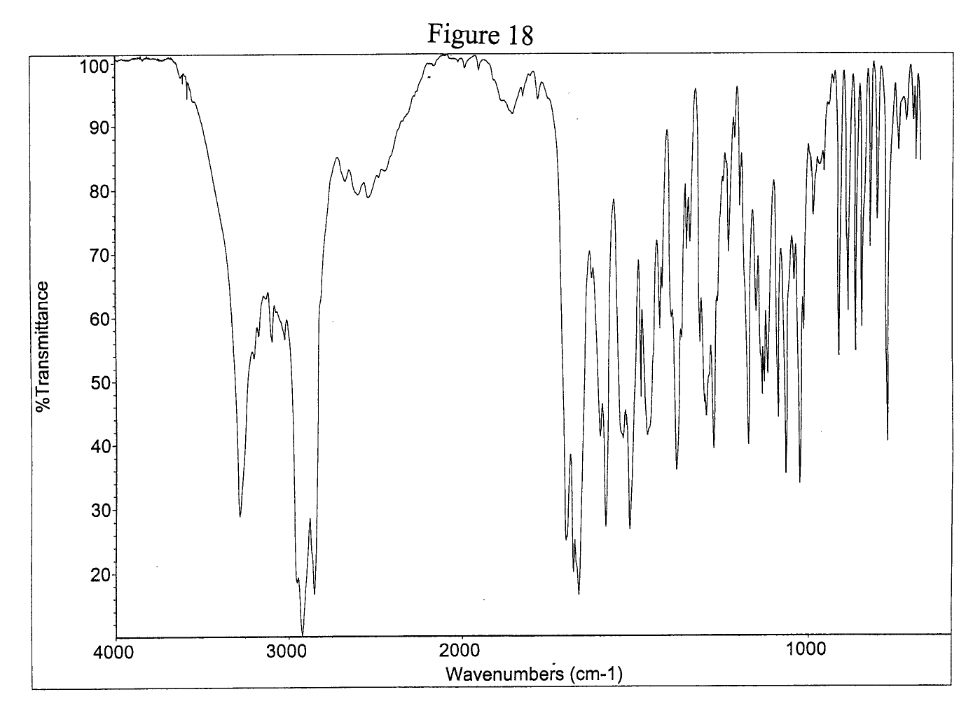

FIG. 19 shows a DSC (Differential Scanning Calorimetry) curve of a crystalline form, consisting of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide} and acetic acid. Crystalline forms and amorphous forms of the present invention can be prepared, for example, by the general preparation procedures described below.

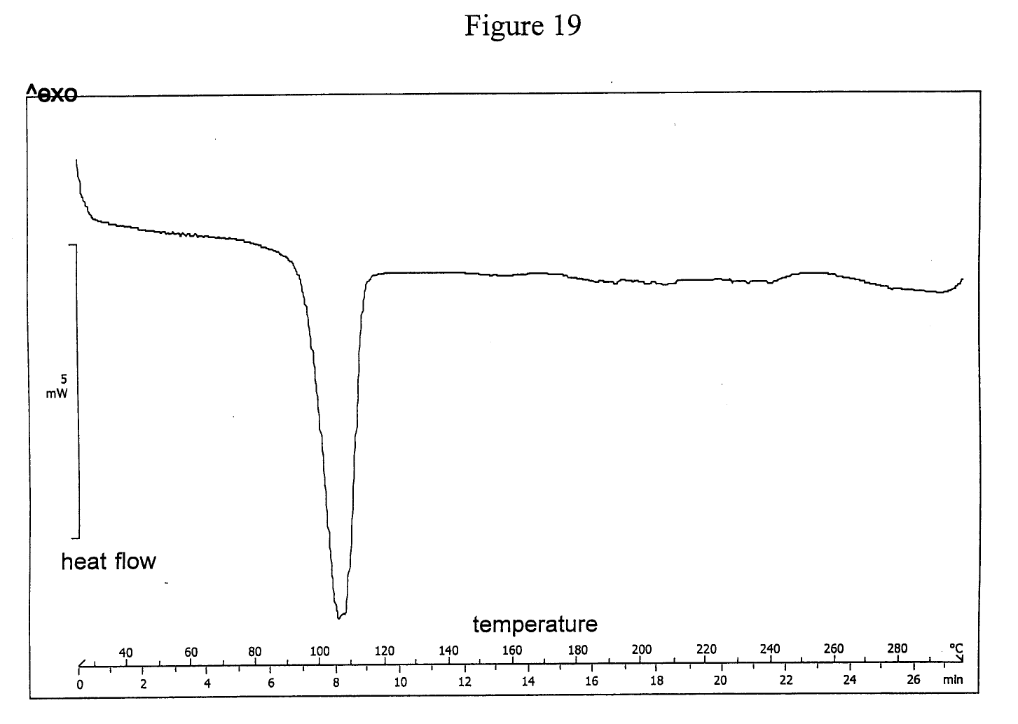

### DETAILED DESCRIPTION OF THE INVENTION

Unless otherwise indicated, the following definitions are set forth to illustrate and define the meaning and scope of the various terms used to describe the invention herein.

“Amorphous forms” or “amorphous” denote a material that lacks long range order and as such does not show a Bragg diffraction peak. The XRPD pattern of an amorphous material is also characterized by one or more amorphous halos.

Bragg's law describes the diffraction of crystalline material with the equation: 2 d sin theta=n lambda, wherein d=perpendicular distance between pairs of adjacent planes in a crystal (d-spacing), theta=Bragg angle, lambda=wavelength and n=integer.

When Bragg's law is fulfilled, the reflected beams are in phase and interfere constructively so that Bragg diffraction peaks are observed in the X-ray diffraction pattern. At angles of incidence other than the Bragg angle, reflected beams are out of phase and destructive interference or cancellation occurs. Amorphous material does not satisfy Bragg's law and no Bragg diffraction peaks are observed in the X-ray diffraction pattern.

“An amorphous halo” is an approximately bell-shaped diffraction maximum in the X-ray powder diffraction pattern of an amorphous substance. The FWHM of an amorphous halo is bigger than two degrees in 2-theta.

“FWHM” means full width at half maximum, which is a width of a peak appearing in an XRPD pattern at its half height.

“(3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}” or “the compound of formula (I)” means the free base of the compounds of formula (I), namely (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}.

“DSC” is used herein as an acronym of Differential Scanning Calorimetry. DCS curves were recorded using a Mettler-Toledo™ differential scanning calorimeter DSC820 or DSC821 with a FRS05 sensor. System suitability tests were performed with Indium as reference substance and calibrations were carried out using Indium, Benzoic acid, Biphenyl and Zinc as reference substances.

For the measurements, approximately 2-6 mg of sample were placed in aluminum pans, accurately weighed and hermetically closed with perforation lids. Prior to measurement, the lids were automatically pierced resulting in approx. 1.5 mm pin holes. The samples were then heated under a flow of nitrogen of about 100 ml/min using heating rates of usually 10 K/min.

For the measurements of amorphous forms, approximately 2-6 mg of sample were placed in aluminum pans, accurately weighed and hermetically closed. The samples were then heated under a flow of nitrogen of about 100 ml/min using heating rates of 10 K/min.

“DVS” is used herein as an acronym of Dynamic Vapor Sorption. DVS isotherms were collected on a DVS-1 (SMS Surface Measurements Systems) moisture balance system. The sorption/desorption isotherms were measured stepwise in a range of 0% RH to 90% RH at 25° C. A weight change of <0.002 mg/min was chosen as criterion to switch to the next level of relative humidity (with a maximum equilibration time of six hours, if the weight criterion was not met). The data were corrected for the initial moisture content of the samples; that is, the weight after drying the sample at 0% relative humidity was taken as the zero point.

“Form A” is used herein as abbreviations for the crystalline form A of the free base of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}.

“Form B” is used herein as abbreviations for the crystalline form B of the free base of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}.

“Free base” is used herein as the abbreviation of the free base of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}.

“IR” is used herein as an acronym of Infra Red spectroscopy. IR spectra were recorded as film of a Nujol suspension of approximately 5 mg of sample and few Nujol between two sodium chloride plates, with an FTIR spectrometer in transmittance. The Spectrometer is a Nicolet™ 20SXB or equivalent (resolution 2 cm⁻¹, 32 or more coadded scans, MCT detector).

“Raman” is used herein as an acronym of Raman spectroscopy. Raman spectra were recorded with a FT-Raman spectrometer (Nicolet Magna IR 860) using the 180-degree reflective configuration. The excitation Nd: YVO4 laser emit at 1064 nm, the beam-splitter is in CaF2 and the detector in InGaAs. Approximately 500 scans are coadded at resolution of 8 cm⁻¹.

“XRPD (is used herein as an acronym of X-Ray Powder Diffraction)” X-ray diffraction patterns were recorded at ambient conditions in transmission geometry with a STOE STADIP diffractometer (Cu K_{α} radiation, primary monochromator, position sensitive detector, angular range 3° to 42° 2 Theta, approximately 60 minutes total measurement time). The samples were prepared and analyzed without further processing (e.g. grinding or sieving) of the substance. “TGA (is used herein as an acronym of ThermoGravimetric Analysis)” was performed on a Mettler-Toledo™ thermogravimetric analyzer (TGA850 or TGA851). System suitability tests and calibrations were carried out according to the internal standard operation procedure.

For the thermogravimetric analyses, approx. 5-10 mg of sample were placed in aluminum pans, accurately weighed and hermetically closed with perforation lids. Prior to measurement, the lids were automatically pierced resulting in approx. 1.5 mm pin holes. The samples were then heated under a flow of nitrogen of about 50 ml/min using a heating rate of 5 K/min.

“Excipient” and “pharmaceutically acceptable excipient” mean inactive pharmaceutically acceptable ingredients that are, other than drug substances, not intended to treat and/or prevent illnesses. It is to be understood that the excipients, including, but not limited to, diluents, surfactants, wetting agents, binders, lubricants, disintegrating agents, carriers, fillers, etc. are of pharmaceutically acceptable grade.

“Pharmaceutically active drug substance(s)” and “drug substance(s)” are used interchangeably to denote a pharmaceutically active principle which is intended to treat and/or prevent illnesses.

“Micronization” means the process whereby the particle size of a single drug substance, is diminished by the aid of a suitable mill, e.g. an air-jet mill.

“Co-micronization” means that a mixture comprising at least one drug substance and at least one excipient is micronized in a suitable mill to obtain a diminished particle size of the drug substance.

“A therapeutically effective amount” of a compound means an amount of compound that is effective to prevent, alleviate or ameliorate symptoms of disease or prolong the survival of the subject being treated. Determination of a therapeutically effective amount is within the skill in the art. The therapeutically effective amount or dosage of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 0.1 mg to about 5,000 mg, 1 mg to about 1,000 mg, or 1 mg to 100 mg may be appropriate, although the upper limit may be exceeded when indicated. The daily dosage can be administered as a single dose or in divided doses, or for parenteral administration, it may be given as continuous infusion.

“Pharmaceutically acceptable carrier” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention are contemplated. Supplementary active compounds can also be incorporated into the compositions.

### General Preparation Procedures

### Preparation of form A of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}

Form A may be formed upon spontaneous or seeded solution mediated phase transformation or upon spontaneous or seeded crystallization in organic solvents such as ethanol, acetonitrile, 2-butanone, ethyl acetate, methyl acetate, isopropyl acetate, tetrahydrofurane, 2-methyl-tetrahydrofurane and others eventually mixed with n-heptane, methylcyclohexane, diethylether, di-isopropylether, dibutylether, tertbutylmethylether or other low polarity solvents or water. Form A is obtained after drying. The accessibility may be influenced by the impurity profile of the compound and the choice of solvent.

### Preparation of form B of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}

Form B may be formed upon spontaneous or seeded solution mediated phase transformation or upon spontaneous or seeded crystallization in solvents such as methanol, ethanol, 1-propanol, 2-propanol, acetonitrile or other solvents eventually mixed with liquids such as n-heptane, methylcyclohexane, diethylether, di-isopropylether, dibutylether, tertbutylmethylether or other low polarity solvents or water, preferably methanol mixed with diisopropylether.

### Preparation of a crystalline form consisting of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide} and acetic acid

This crystalline form can be produced by digestion in solvents as e.g. ethanol and water. It can also be prepared by re-crystallization of form A, B or amorphous form of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide} and acetic acid with or without seeding in solvent systems comprising but not limited to ethanol.

### Preparation of the amorphous form of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}

Evaporation of a solution of (3R,4R)-1-(2,2-Difluoroethyl)pyrrolidine-3,4-dicarboxylic acid 3-[(5-chloro-pyridin-2-yl)-amide] 4-{[2-fluoro-4-(2-oxo-2H-pyridin-1-yl)-phenyl]-amide} in organic_solvents such as dichloromethane, ethyl acetate or others leading to amorphous solid state usually as a foam.

The crystalline form(s) and the amorphous forms of the present invention can be used as medicaments, e.g. in the form of pharmaceutical compositions or preparations for enteral, parenteral or topical administration. They can be administered, for example, perorally, e.g. in the form of tablets, coated tablets, dragées, hard and soft gelatine capsules, solutions, emulsions or suspensions, rectally, e.g. in the form of suppositories, parenterally, e.g. in the form of injection solutions or suspensions or infusion solutions, or topically, e.g. in the form of ointments, creams or oils. Oral administration is preferred.

The production of the pharmaceutical compositions or preparations can be effected in a manner which will be familiar to any person skilled in the art by bringing the described crystalline forms or the amorphous of the compounds of formula (I), optionally in combination with other therapeutically valuable substances, into a galenical administration form together with suitable, non-toxic, inert, therapeutically compatible solid or liquid carrier materials and, if desired, usual pharmaceutical adjuvants.

Suitable carrier materials are not only inorganic carrier materials, but also organic carrier materials. Thus, for example, lactose, corn starch or derivatives thereof, talc, stearic acid or its salts can be used as carrier materials for tablets, coated tablets, dragées and hard gelatine capsules. Suitable carrier materials for soft gelatine capsules are, for example, vegetable oils, waxes, fats and semi-solid and liquid polyols (depending on the nature of the active ingredient no carriers might, however, be required in the case of soft gelatine capsules). Suitable carrier materials for the production of solutions and syrups are, for example, water, polyols, sucrose, invert sugar. Suitable carrier materials for injection solutions are, for example, water, alcohols, polyols, glycerol and vegetable oils. Suitable carrier materials for suppositories are, for example, natural or hardened oils, waxes, fats and semi-liquid or liquid polyols. Suitable carrier materials for topical compositions are glycerides, semi-synthetic and synthetic glycerides, hydrogenated oils, liquid waxes, liquid paraffins, liquid fatty alcohols, sterols, polyethylene glycols and cellulose derivatives.

Usual stabilizers, preservatives, wetting and emulsifying agents, consistency-improving agents, flavour-improving agents, salts for varying the osmotic pressure, buffer substances, solubilizers, colorants and masking agents and antioxidants come into consideration as pharmaceutical adjuvants.

The dosage of the described crystalline forms or the amorphous of the compounds of formula (I) can vary within wide limits depending on the disease to be controlled, the age and the individual condition of the patient and the mode of administration, and will, of course, be fitted to the individual requirements in each particular case. For adult patients a daily dosage of about 1 to 1000 mg, especially about 1 to 300 mg, comes into consideration. Depending on severity of the disease and the precise pharmacokinetic profile the crystalline forms or amorphous forms of the present invention could be administered with one or several daily dosage units, e.g. in 1 to 3 dosage units.

The pharmaceutical compositions or preparations conveniently contain about 1-500 mg, preferably 1-100 mg, of the crystalline form(s) or the amorphous forms of the compound of formula (I).

The use of the described crystalline forms or the amorphous compounds of formula (I) are for the treatment and/or prophylaxis of diseases which are associated with coagulation factor Xa, particularly as therapeutically active substances for the treatment and/or prophylaxis of thrombotic disorders, arterial thrombosis, venous thrombosis, deep vein thrombosis, peripheral arterial occlusive disease, unstable angina pectoris, myocardial infarction, coronary artery disease, pulmonary embolism, stroke due to atrial fibrillation, inflammation, arteriosclerosis, acute vessel closure associated with thrombolytic therapy or restenosis, and/or tumor.

Another use of the described crystalline forms or the amorphous compounds of formula (I) are for the preparation of pharmaceutical compositions for the therapeutic and/or prophylactic treatment of diseases which are associated with coagulation factor Xa, particularly for the therapeutic and/or prophylactic treatment of thrombotic disorders, arterial thrombosis, venous thrombosis, deep vein thrombosis, peripheral arterial occlusive disease, unstable angina pectoris, myocardial infarction, coronary artery disease, pulmonary embolism, stroke due to atrial fibrillation, inflammation, arteriosclerosis, acute vessel closure associated with thrombolytic therapy or restenosis, and/or tumor.

To prepare the pharmaceutical compositions, containing the crystalline form(s) or the amorphous forms of the compound of formula (I), these materials are often micronized. Micronization is a commonly used and well known process in the pharmaceutical industry to reduce the particle size of drug substances. The reason for micronization is usually to increase the bioavailability of the drug substance or to improve its overall technical processability. Micronization of crystalline form B of (3R,4R)-1-(2,2-Difluoro-ethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide} is however not feasible, due to an increased electrostatic charging of the drug substance during milling which gives rise to increased stickiness of the particles of the drug substance to each other and to the inner walls of the mill, thereby plugging the mill. This problem may be overcome by the addition of a pharmaceutical excipient, which is known to be easily micronized, to the drug substance to form a suitable mixture and then micronize this mixture to diminish the particle size of the drug substance. This process is also referred to as “co-micronization”. A well known and for co-micronization suitable excipient is lactose which is available on the market in various micronized forms. But also several other excipient are known to be suitable for co-micronization, for example sugars and sugar alcohols like trehalose, mannitol, xylitol and sorbitol.

### EXAMPLES

The following Examples serve to illustrate the present invention in more detail. They are, however, not intended to limit its scope in any manner.

### Example 1

### Preparation of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}

### Step 1: [3+2] Cycloaddition

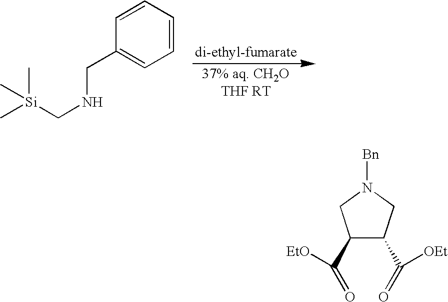

900 g of N-Benzyl-N-trimethylsilylmethyl-amine (4654 mmol) were dissolved in 5.6 l THF at 20-25° C. 450 ml of 36% aqueous formaldehyde (5880 mmol, 1.26 equiv.) were added over 15 min, keeping the temperature between 20-25° C. After 15 min, a mixture of 760 ml diethyl fumarate (1 equiv.), 2.25 l THF and 11.2 ml trifluoroacetic acid (0.03 equiv.) was added over 15 min. The reaction mixture was stirred overnight keeping the temperature between 20-30° C. (in process control by GC). 3.5 l of 1N HCl were added, followed by 2.3 l heptane. The aqueous phase was separated and washed with 3.4 l heptane. The heptane phases were washed sequentially with 3.5 l 1N HCl. 4.5 l MTBE were added to the combined aqueous phases. 720 ml of 32% NaOH_{aq} were added (pH 13) under vigorous stirring. The aqueous phase was separated and re-extracted with 4.5 l MTBE. The MTBE phases were washed sequentially with 2.2 l water, combined and concentrated to dryness at 45° C. to give 1.295 kg of crude (rac)-trans-N-benzyl-pyrrolidine-3,4-dicarboxylic acid diethyl ester. If required, the crude cycloadduct can be distilled.

### Step 2-3: De-Benzylation/Boc Protection

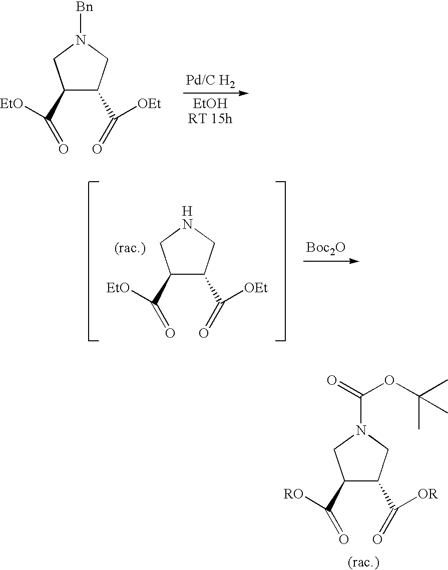

1.295 kg (rac)-trans-1-Benzyl-pyrrolidine-3,4-dicarboxylic acid diethyl ester were hydrogenated at room temperature, in 6.5 l EtOH with 100 g, 10% Pd/C catalyst. After completion of the reaction, the catalyst was filtered and a solution of 935 g, di-t-butyl-dicarbonate (1.01 equiv) in 480 ml EtOH was added. After completion of the reaction (in process control by GC), the reaction mixture was evaporated, dissolved in 9.7 l THF. 8 ml water were added, followed by 5.3 g, DMAP (0.01 equiv.). The reaction mixture was stirred 30 min. at room temperature and concentrated to dryness. The residue was dissolved in 6.5 l MTBE, washed with 1.29 l 5% aqueous citric acid solution, 3.3 l 10% aqueous NaHCO₃ solution and 3.3 l water. The organic phases were washed sequentially with 6.5 l MTBE. The combined organic phases were dried over Na₂SO₄ and concentrated to dryness at 40° C. to give 1.233 kg of crude (rac)-trans-N-Boc-pyrrolidine-3,4-dicarboxylic acid diethyl ester.

### Step 4: Enzymatic Resolution

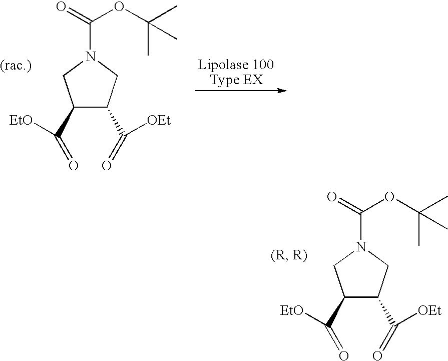

32 g (rac)-trans-N-Boc-pyrrolidine-3,4-dicarboxylic acid diethyl ester (96.19 mmol, 95a % GC) were emulsified under vigorous stirring in 32 ml heptane and 256 ml 0.1M sodium phosphate buffer pH 7.0. The emulsion was cooled to 0-1° C. 2.30 ml Novozyme Lipolase 100 L Type EX were added and the pH kept constant at 7.0 by the automated addition (pH-stat) of 1.0M NaOH-solution. After reaching the targeted enantiomeric excess, (typically >99%, ca 45 h reaction time, 0.55 equiv. NaOH added, GC in process control), 250 ml dichloromethane were added. The aqueous phase was separated and extracted twice with 500 ml dichloromethane. The combined organic phases were evaporated during which a white precipitate was formed. The residue was re-dissolved in 250 ml ethyl acetate and the white precipitate was filtered off. The filtrate was washed with 75 ml saturated aqueous sodium bicarbonate solution. The organic phase was dried over sodium sulfate, evaporated and dried under high vacuum overnight to give 13.47 g, (3R,4R)-trans-N-Boc-pyrrolidine-3,4-dicarboxylic acid diethyl ester as a light yellow oil (96% GC).

The product can alternatively be extracted with heptane or MTBE, preferably heptane. NaCl can also be added to the aqueous phase to facilitate the phase separations.

### Step 5: Selective Monohydrolysis

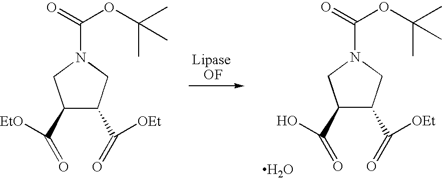

2.95 kg, (3R,4R)-trans-N-Boc-pyrrolidine-3,4-dicarboxylic acid diethyl ester were stirred in 26.5 l of an aqueous KPI 5 mM/1M D-glucose to form an emulsion. 5.9 g of Amano Lipase OF dissolved in 0.5 l water were added. The pH was kept at 7.2 by addition of 1M NaOH. After completion of the reaction (8.4 kg, 1M NaOH, 24 h reaction time, GC in process control), the reaction was stopped by addition of 10 l MTBE. The organic layer was separated and discarded. 40 l ethyl acetate were added and the pH was adjusted to 4 by addition of H₂SO₄. The organic layer was separated and the aqueous phase was re-extracted with 40 l ethyl acetate. The combined organic phases were evaporated to dryness to give 2.35 kg of (3R,4R)-trans-N-Boc-pyrrolidine-3,4-dicarboxylic acid monoethyl ester.

### The (3R,4R)-trans-N-Boc-pyrrolidine-3,4-dicarboxylic acid monoethyl ester can be crystallized in acetone/water

3.2 kg of (3R,4R)-trans-N-Boc-pyrrolidine-3,4-dicarboxylic acid monoethyl ester were dissolved in 3.2 l acetone. To this solution, 3.2 l of 0.1% aqueous acetic acid solution were added at room temperature. The turbid solution was seeded. The crystallization started after 15 min. After an additional 30 min., 30 l water were added and the suspension was stirred 22 h at room temperature. The suspension was filtered. The filter cake was washed in portions with water, in total 7 l and was dried to constant weight to give 3.295 kg of (3R,4R)-trans-N-Boc-pyrrolidine-3,4-dicarboxylic acid monoethyl ester monohydrate as a white powder.

### Step 6: First Amide Coupling

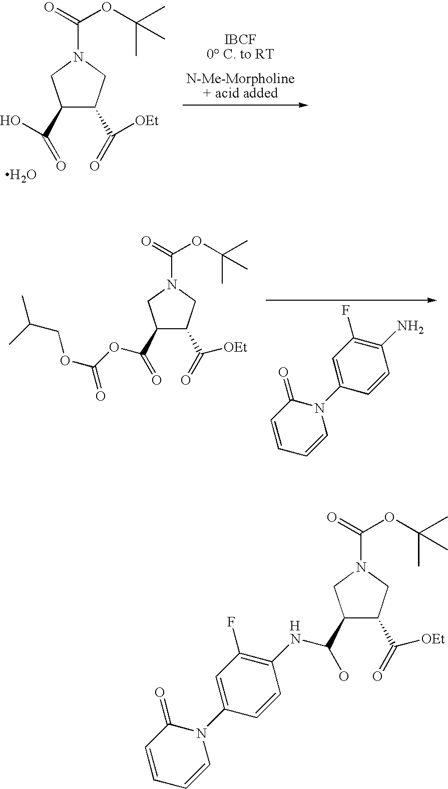

135 g, trans-N-Boc-pyrrolidine-3,4-dicarboxylic acid monoethyl ester monohydrate were suspended in 700 ml toluene and concentrated to 150-200 ml (60° C. jacket temperature under ca 100 mbar; azeotropic removal of water, residual water content checked by Karl Fischer analysis). 400 ml THF were added followed by 55 ml N-methylmorpholine (1.11 equiv). The resulting solution was added over 30 min. to a cold (0-5° C.) solution of 60 ml (1.04 equiv.) isobutyl chloroformiate in 900 ml THF. The addition funnel was washed with 50 ml THF. The white suspension was stirred 15 min. at 0-5° C. 90 g of the fluoroaniline (1.0 equiv.) were added in one portion and the reaction mixture was heated at reflux. After completion of the reaction (HPLC in process control), the reaction mixture was cooled to RT. 900 ml toluene were added followed by 500 ml 1M HCl. The aqueous phase was separated and extracted with 900 ml toluene. The organic phases were washed sequentially with 500 ml HCl 1M and 500 ml 5% aqueous NaHCO₃ solution. The organic phases were combined, dried over Na₂SO₄ and concentrated to ca 500 ml (60° C. jacket temperature). The isobutanol was removed by azeotropic distillation at constant volume with ca 1 l toluene (isobutanol removal checked by GC). The crude product solution was then concentrated to 337 g (60% m/m solution in toluene which was used directly in the next step, corresponds to 97% yield).

### Step 7: Second Amide Coupling

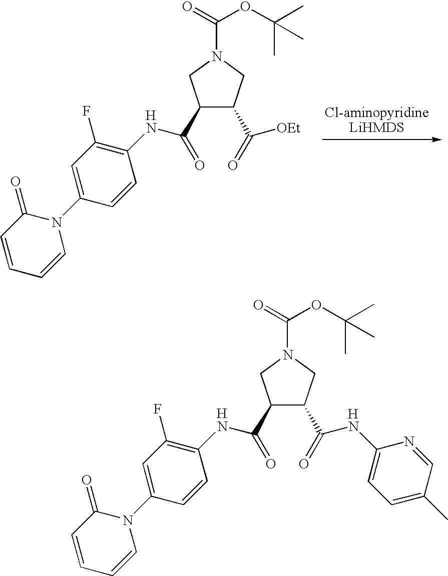

337 g of a 60% m/m of the amide ester (see previous step) solution in toluene (431 mmol, 1 equiv.) was charged in the reactor, followed by 650 ml THF. 86 g, 5-chloro-2-aminopyridine (1.5 equiv.) were added. 1.2 L of 1M LiHMDS solution in THF was added over 30 min. keeping the temperature between 20-25° C. After completion of the reaction (HPLC in process control), a solution consisting of 300 ml 37% HCl_{aq} in 1.2 l water was added (pH 1-2). 2 l dichloromethane were added and the organic phase was separated and washed with 1 l water. The aqueous phases were extracted sequentially with 1 l dichloromethane. The combined dichloromethane phases were concentrated to a volume of 2.5-3.5 l. A solvent exchange to ethanol was performed at constant volume (60° C. jacket temperature, 400 to 100 mbar, 5 l Ethanol in total) during which crystallization starts. The suspension was cooled to RT, stirred overnight at RT and 2 h at 0-5° C. The suspension was filtered and the filter cake was washed 4 times with 250 ml cold (−20° C.) EtOH. The crystals were dried at 45° C. to constant weight to give 180 g of the expected Boc-pyrrolidine bis-amide as a white powder (75% yield).

### Example 2

### Preparation of Crystalline Form A of the Compound of Formula (I)

53.2 g of 3-(5-Chloro-pyridin-2-ylcarbamoyl)-4-[2-fluoro-4-(2-oxo-2H-pyridin-1-yl)-henylcarbamoyl]-pyrrolidine-1-carboxylic acid tert-butyl ester (95.7 mmol, 1 equiv.) were added at room temperature, in portions to a solution consisting of 160 ml water and 160 ml 37% HCl_{aq} (20 equiv.). After completion of the reaction (ca 30 min., HPLC in process control), the resulting solution was added over 1 h to a hot (50° C.) solution consisting of 197 g sodium bicarbonate (24.5 equiv.), 320 ml water, 530 ml ethyl acetate and 23 g, 2,2-difluoroethyl triflate (1.1 equiv.). The addition funnel was washed with 15 ml water. After completion of the reaction (ca 30 min., HPLC in process control), the reaction mixture was cooled to RT. The aqueous phase was separated and re-extracted with 530 ml ethyl acetate. The organic phases were washed sequentially with 265 ml half saturated NaCl solution. The combined ethyl acetate phases were dried over Na₂SO₄ and filtered. The Na₂SO₄ filter cake was washed with 230 ml ethyl acetate. The filtrate was concentrated to 1 l and a solvent exchange to ethanol was performed (constant volume, 60° C. jacket temperature, ca 2 l, ethanol used). The hot solution was cooled to RT and seeded with form A upon which the crystallization started. After stirring overnight at room temperature, the white suspension was cooled to −20° C. After 1 h at −20° C., the suspension was filtered and washed in portions with in total 100 ml cold (−20° C.) ethanol. The crystals were dried to constant weight (50° C./reduced pressure) to give 40 g of a white powder (78% yield).

### Form A Seeds Preparation

Form A seeding crystals can be prepared by spontaneous crystallization at approx. 0° C. of solutions prepared by dissolving approx. 0.5 g of (3R,4R)-1-(2,2-Difluoroethyl)pyrrolidine-3,4-dicarboxylic acid 3-[(5-chloro-pyridin-2-yl)-amide] 4-{[2-fluoro-4-(2-oxo-2H-pyridin-1-yl)-phenyl]-amide} in organic solvents as tetrahydrofurane (approx. 1.8 g) or 2-butanone (approx. 2.7 g) or ethyl acetate (approx. 3.9 g) with subsequent filtration and drying.

### Characterization of Form A

Form A can be characterized:

by an X-ray powder diffraction pattern obtained with a Cu Kα radiation having characteristic peaks expressed in degrees 2 Theta at approximately: 5.4, 8.3, 9.9, 10.8, 14.4, 16.6, 18.6, 19.9, 21.0, 21.7, 22.9 and 26.0. The term “approximately” means in this context that there is an uncertainty in the measurements of the degrees 2 Theta of ±0.2 (expressed in degrees 2 Theta).

by an infrared spectrum having sharp bands at approximately: 3256, 1665, 1624, 1608, 1591, 1575, 1526, 1460, 1429, 1377, 1341, 1292, 1175, 1147, 1119, 1061, 1034, 1013, 914, 900, 835, 761, and 643 cm⁻¹. The term “approximately” means in this context that there is an uncertainty in the measurements of the wavenumbers of ±3 cm⁻¹.

by a Raman spectrum having sharp bands at approximately: 3086, 2972, 1668, 1625, 1590, 1576, 1535, 1387, 1312, 1227, 1214, 1115, 1032, 917, 841, 689 and 268 cm⁻¹. The term “approximately” means in this context that there is an uncertainty in the measurements of the Raman shift of ±3 cm⁻¹.

by a melting point with onset temperature (DSC) in the range of about 100° C. to 105° C.

### Example 3

### Preparation of Crystalline Form B of the Compound of Formula (I)

750 g of form A of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide} were suspended in a mixture of methanol (3.4 l) and di-isopropylether (5.7 l) at ambient temperature. The suspension was heated to ca. 34° C. and stirred until a solution was obtained. The solution was seeded with form B of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide} and cooled to 25° C. within 1 h and stirred at that temperature for 1 h. The suspension was heated to 35° C. for 1 h, cooled to 25° C. within 1 h, cooled to 20° C. within 12 h and stirred at that temperature overnight. The suspension was filtered. The reactor was washed with 2.5 l of the mother liquor. The filter cake was washed with a cold (0° C.) mixture of methanol (250 ml) and di-isopropylether (500 ml). The crystals were dried at 50° C. under vacuum. Yield: 600 g.

### Form B Seeds Preparation

Form B seeding crystals can be prepared upon aging of solid (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide} in organic solvents as methanol, ethanol, 1-propanol, 2-propanol and others at varying temperatures (e.g. 0-50° C.) for an appropriate time (e.g. several days).

### Characterization of Form B

Form B is a solvent free form and no significant weight loss is normally observed in the TGA curve prior to decomposition and Form B can be characterized:

by an X-ray powder diffraction pattern obtained with a Cu Kα radiation having characteristic peaks expressed in degrees 2 Theta at approximately: 7.4, 8.6, 9.4, 11.4, 15.0, 17.2, 17.8, 18.3, 20.7 and 27.8. The term “approximately” means in this context that there is an uncertainty in the measurements of the degrees 2 Theta of ±0.2 (expressed in degrees 2 Theta).

by an infrared spectrum having sharp bands at approximately: 3287, 1665, 1589, 1577, 1518, 1430, 1377, 1334, 1289, 1246, 1210, 1174, 1145, 1117, 1064, 1029, 1017, 1010, 906, 873, 864, 841, 830, 775, 759, 734, and 708 cm⁻¹. The term “approximately” means in this context that there is an uncertainty in the measurements of the wavenumbers of ±3 cm⁻¹.

by a Raman spectrum having sharp bands at approximately: 3287, 3072, 2961, 2828, 1673, 1626, 1590, 1536, 1386, 1313, 1258, 1212, 1115, 1030, 841, 689, 631, 560, 449 and 207 cm⁻¹. The term “approximately” means in this context that there is an uncertainty in the measurements of the Raman shift of ±3 cm⁻¹.

by a melting point with onset temperature (DSC) in the range of about 140° C. to 155° C.

### Example 4

### Preparation of a crystalline form, consisting of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide} and acetic acid

100 mg of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide} and 57 mg Acetic acid (99.5%, puriss. pa) (1 part (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}+5 part Acetic acid) were dissolved in 0.25 ml ethanol and heated up to dissolve both substances. The clear solution was cooled down to room temperature without stirring. After 26 days the crystals were filtered and dried at room temperature for 14 h.

### Characterization

This crystalline form can be characterized:

by an X-ray powder diffraction pattern obtained with a Cu Kα radiation having characteristic peaks expressed in degrees 2 Theta at approximately: 7.6, 11.9, 12.8, 13.2, 16.8, 18.5, 19.0, 19.5, 19.8, 20.5, 20.8, 23.2, 25.6, 26.3. The term “approximately” means in this context that there is an uncertainty in the measurements of the degrees 2 Theta of ±0.2 (expressed in degrees 2 Theta).

by an infrared spectrum having sharp bands at approximately: 3284, 3097, 1700, 1679, 1663, 1602, 1585, 1536, 1517, 1485, 1429, 1422, 1314, 1297, 1275, 1231, 1176, 1151, 1135, 1129, 1119, 1089, 1067, 1027, 914, 887, 866, 848, 824 and 775 cm⁻¹. The term “approximately” means in this context that there is an uncertainty in the measurements of the wavenumbers of ±3 cm⁻¹.

### Example 5

### Preparation of the Amorphous Form of the Compound of Formula (I)

### Evaporation

2.99 g of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide} was dissolved in dichloromethane (21 ml). Dichloromethane was evaporated at ca. 44° C. under vacuum yielding foam which was dried at 50° C. and 5-20 mbar for 4 days.

### Characterization of the Amorphous Form

The amorphous form can be characterized:

by the lack of a Bragg diffraction peak in its XRPD pattern.

by an infrared spectrum having sharp bands at approximately: 3240, 1696, 1660, 1575, 1524, 1458, 1428, 1376, 1329, 1290, 1171, 1113, 1035, 1009, 911, 839, 764, and 632 cm⁻¹. The term “approximately” means in this context that there is an uncertainty in the measurements of the wavenumbers of ±3 cm⁻¹.

by a Raman spectrum having sharp bands at approximately: 3093, 2975, 1693, 1625, 1575, 1535, 1455, 1386, 1317, 1228, 1128, 1114, 845, 687, 632, 566 and 529 cm⁻¹. The term “approximately” means in this context that there is an uncertainty in the measurements of the Raman shift of ±3 cm⁻¹.

by a glass transition temperature (DSC) in the range of approximately 58° C. to 85° C. (The glass transition temperature is largely dependant on the water/solvent content).

### Example 6

### Preparation of Co-Micronized Form B for Use in Pharmaceutical Formulations

1. Production of the mixture for co-micronization: Prepare a mixture comprising predefined amounts of excipient (e.g.) lactose and form B in an appropriate mixing vessel by mixing for 6 min. (Tumble-Mixer), sieving through a 2 mm mesh size and repeating the mixing for another 6 min (Tumble-Mixer).

2. Co-micronization: The resulting mixture is then co-micronized using a standard jet mill (standard conditions depending on scale).

3. Final Mixing: The co-micronized material was finally mixed for another 3 min (Tumble-Mixer).

Co-micronized mixtures containing 14.5%, 29.1%, 33.3% and 65.8% by weight of form B and the corresponding amount of lactose were then used for the manufacturing of pharmaceutical formulations as further described.

### Example 7

### Stability of Crystalline Form B

No significant degradation could be observed after storage for 1 year up to 40° C./75% rh and the crystal form B has not been changed when compared to the initial analysis. The Form B has been characterized with IR and XRPD. The chemical stability was measured by HPLC (high performance liquid chromatography).

### Example A

Film coated tablets containing the following ingredients can be manufactured in a conventional manner:

| Ingredients | Per tablet | | | |
|---|---|---|---|---|
| Kernel: | | | | |
| Form A or form B of the compound of | 10.0 | mg | 200.0 | mg |
| formula (I) | | | | |
| Microcrystalline cellulose | 23.5 | mg | 43.5 | mg |
| Lactose | 60.0 | mg | 70.0 | mg |
| Povidone K30 | 12.5 | mg | 15.0 | mg |
| Sodium starch glycolate | 12.5 | mg | 17.0 | mg |
| Magnesium stearate | 1.5 | mg | 4.5 | mg |
| (Kernel Weight) | 120.0 | mg | 350.0 | mg |
| Film Coat: | | | | |
| Hydroxypropyl methyl cellulose | 3.5 | mg | 7.0 | mg |
| Polyethylene glycol 6000 | 0.8 | mg | 1.6 | mg |
| Talc | 1.3 | mg | 2.6 | mg |
| Iron oxyde (yellow) | 0.8 | mg | 1.6 | mg |
| Titan dioxide | 0.8 | mg | 1.6 | mg |

The active ingredient is sieved and mixed with microcristalline cellulose and the mixture is granulated with a solution of polyvinylpyrrolidon in water. The granulate is mixed with sodium starch glycolate and magesiumstearate and compressed to yield kernels of 120 or 350 mg respectively. The kernels are lacquered with an aqueous solution/suspension of the above mentioned film coat.

### Example B

Capsules containing the following ingredients can be manufactured in a conventional manner:

| Ingredients | Per capsule |
|---|---|
| Form A or form B of the compound of formula (I) | 25.0 mg |
| Lactose | 150.0 mg |
| Maize starch | 20.0 mg |
| Talc | 5.0 mg |

The components are sieved and mixed and filled into capsules of size 2.

### Example C

Injection solutions can have the following composition:

| Form A or form B of the compound of formula (I) | 3.0 | mg |
|---|---|---|
| Polyethylene Glycol 400 | 150.0 | mg |
| Acetic Acid | q.s. ad pH 5.0 | |
| Water for injection solutions | ad 1.0 | ml |

The active ingredient is dissolved in a mixture of Polyethylene Glycol 400 and water for injection (part). The pH is adjusted to 5.0 by Acetic Acid. The volume is adjusted to 1.0 ml by addition of the residual amount of water. The solution is filtered, filled into vials using an appropriate overage and sterilized.

### Example D

Soft gelatin capsules containing the following ingredients can be manufactured in a conventional manner:

| Capsule contents | | |
|---|---|---|
| Form A or form B of the compound of formula (I) | 5.0 | mg |
| Yellow wax | 8.0 | mg |
| Hydrogenated Soya bean oil | 8.0 | mg |
| Partially hydrogenated plant oils | 34.0 | mg |
| Soya bean oil | 110.0 | mg |
| Weight of capsule contents | 165.0 | mg |
| Gelatin capsule | | |
| Gelatin | 75.0 | mg |
| Glycerol 85% | 32.0 | mg |
| Karion 83 | 8.0 | mg (dry matter) |
| Titan dioxide | 0.4 | mg |
| Iron oxide yellow | 1.1 | mg |

The active ingredient is dissolved in a warm melting of the other ingredients and the mixture is filled into soft gelatin capsules of appropriate size. The filled soft gelatin capsules are treated according to the usual procedures.

### Example E

Sachets containing the following ingredients can be manufactured in a conventional manner:

| | |
|---|---|
| Form A or form B of the compound of formula (I) | 50.0 mg |
| Lactose, fine powder | 1015.0 mg |
| Microcristalline cellulose (AVICEL PH 102) | 1400.0 mg |
| Sodium carboxymethyl cellulose | 14.0 mg |
| Polyvinylpyrrolidon K 30 | 10.0 mg |
| Magnesiumstearate | 10.0 mg |
| Flavoring additives | 1.0 mg |

The active ingredient is mixed with lactose, microcristalline cellulose and sodium carboxymethyl cellulose and granulated with a mixture of polyvinylpyrrolidon in water. The granulate is mixed with magnesiumstearate and the flavouring additives and filled into sachets.

Unless stated to the contrary, all compounds in the examples were prepared and characterized as described. All ranges recited herein encompass all combinations and subcombinations included within that range limit. All patents and publications cited herein are hereby incorporated by reference in their entirety.

1. Crystalline form A of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}, which is characterized by an X ray powder diffraction pattern comprising at least three of 2[theta] values selected from the group consisting of approximately 5.4, approximately 8.3, approximately 9.9, approximately 10.8, approximately 14.4, approximately 16.6, approximately 18.6, approximately 19.9, approximately 21.0, approximately 21.7, approximately 22.9 and approximately 26.0.

2. Crystalline form A according to claim 1, wherein the X ray powder diffraction pattern comprises at least five of 2[theta] values selected from the group consisting of approximately 5.4, approximately 8.3, approximately 9.9, approximately 10.8, approximately 14.4, approximately 16.6, approximately 18.6, approximately 19.9, approximately 21.0, approximately 21.7, approximately 22.9 and approximately 26.0.

3. Crystalline form A according to claim 1, wherein the X ray powder diffraction pattern comprises at least seven of 2[theta] values selected from the group consisting of approximately 5.4, approximately 8.3, approximately 9.9, approximately 10.8, approximately 14.4, approximately 16.6, approximately 18.6, approximately 19.9, approximately 21.0, approximately 21.7, approximately 22.9 and approximately 26.0.

4. Crystalline form B of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}, which is characterized by an X ray powder diffraction pattern comprising at least three of 2[theta] values selected from the group consisting of approximately 7.4, approximately 8.6, approximately 9.4, approximately 11.4, approximately 15.0, approximately 17.2, approximately 17.8, approximately 18.3, approximately 20.7 and approximately 27.8.

5. Crystalline form B according to claim 4, wherein the X ray powder diffraction pattern comprises at least five of 2[theta] values selected from the group consisting of approximately 7.4, approximately 8.6, approximately 9.4, approximately 11.4, approximately 15.0, approximately 17.2, approximately 17.8, approximately 18.3, approximately 20.7 and approximately 27.8.

6. Crystalline form B according to claim 4, wherein the X ray powder diffraction pattern comprises at least seven of 2[theta] values selected from the group consisting of approximately 7.4, approximately 8.6, approximately 9.4, approximately 11.4, approximately 15.0, approximately 17.2, approximately 17.8, approximately 18.3, approximately 20.7 and approximately 27.8.

7. A crystalline form, consisting essentially of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide} and acetic acid.

8. An amorphous form of (3R,4R)-1-(2,2-Difluoroethyl)-pyrrolidine-3,4-dicarboxylic acid 3-[(5-chloro-pyridin-2-yl)-amide] 4-{[2-fluoro-4-(2-oxo-2H-pyridin-1-yl)-phenyl]-amide}, which is characterized by an X ray powder diffraction pattern lacking a Bragg diffraction peak.

9. A pharmaceutical composition comprising a therapeutically effective amount of the crystalline form A of claim 1 and a pharmaceutically acceptable carrier.

10. A pharmaceutical composition comprising a therapeutically effective amount of the crystalline form B of claim 4 and a pharmaceutically acceptable carrier.

11. A pharmaceutical composition comprising a therapeutically effective amount of the amorphous form of claim 8 and a pharmaceutically acceptable carrier.
